(19) United States
(12) Patent Application Publication
(10) Pub. No.: US 2018/0064620 A1
(43) Pub. Date: Mar. 8, 2018

(54) COSMETIC COMPOSITION COMPRISING AT LEAST ONE ORGANOSILICON COMPOUND, AT LEAST ONE ANIONIC SURFACTANT AND AT LEAST ONE NONIONIC THICKENER, AND PROCESS USING THE COMPOSITION

(71) Applicant: L'OREAL, Paris (FR)

(72) Inventors: Valérie VIRAVAU, Paris (FR); Carine AIRES, Paris (FR)

(21) Appl. No.: 15/810,988

(22) Filed: Nov. 13, 2017

Related U.S. Application Data
(63) Continuation of application No. 12/977,117, filed on Dec. 23, 2010.
(60) Provisional application No. 61/298,291, filed on Jan. 26, 2010.

(30) Foreign Application Priority Data
Dec. 23, 2009 (FR) 0959497

(51) Int. Cl. A61K 8/58; A61Q 5/12; A61Q 5/02; A61K 8/87; A61K 8/86; A61K 8/73; A61K 8/39; A61K 8/46; A61K 8/42

(52) U.S. Cl. CPC A61K 8/585; A61Q 5/12; A61Q 5/02; A61K 8/87; A61K 8/86; A61K 8/737; A61K 8/731; A61K 8/466; A61K 8/463; A61K 8/42; A61K 8/39

(57) ABSTRACT

Provided is a detergent and conditioning cosmetic composition for keratin fibers, comprising, in a cosmetically acceptable medium: (i) at least one organosilicon compound chosen from silanes comprising one silicon atom and siloxanes comprising two or three silicon atoms, wherein the at least one organosilicon compound also comprises at least one basic chemical functional group and at least one group chosen from hydroxyl and hydrolysable groups per molecule, wherein the at least one organosilicon compound is present in a total amount ranging from 0.01% to 5% by weight, relative to the total weight of the cosmetic composition; (ii) at least one anionic surfactant, and iii) at least one nonionic thickener, wherein the weight ratio of the at least one organosilicon compound to the at least one anionic surfactant is equal to or greater than 5×10⁻⁴:1. Also provided herein is a process involving said compositions.

This application claims benefit of U.S. Provisional Application No. 61/298,291, filed Jan. 26, 2010. This application also claims benefit of priority under 35 U.S.C. § 119 to French Patent Application No. 0959497, filed Dec. 23, 2009.

Disclosed herein is a cosmetic composition for cleansing and conditioning keratin fibers, for example human keratin fibers such as the hair, comprising, in a cosmetically acceptable medium, at least one organosilicon compound, at least one anionic surfactant and at least one nonionic thickener, wherein the weight ratio of the at least one organosilicon compound to the at least one anionic surfactant is equal to or greater than 5×10⁻⁴:1.

Also disclosed herein is a cosmetic process for treating keratin fibers, employing the cosmetic composition.

It can be a common practice to use detergent compositions (such as shampoos) based, for example, on standard surfactants such as anionic, nonionic and/or amphoteric type, further such as anionic type, for cleansing and/or washing keratin materials such as the hair. These compositions can be applied to wet hair and the lather generated by massaging or frictioning with the hands can make it possible, after rinsing with water, to remove the diverse types of soiling initially present on the hair or the skin.

These detergent compositions can have good washing power, but their intrinsic cosmetic properties however may remain quite poor, for example, due to the fact that the relatively aggressive nature of such a cleansing treatment may in the long term give rise to more or less pronounced damage on hair fibers, associated, for example, with the gradual removal of the fats or proteins contained in or at their surface.

Thus, to improve the cosmetic properties of the above detergent compositions, and for example, of those that can be applied to sensitized hair (i.e. hair that may be damaged or embrittled by the action of external atmospheric agents such as light and/or bad weather, and/or mechanical or chemical treatments such as blow-drying, combing, dyeing, bleaching, permanent-waving and/or relaxing), it may now become common practice to introduce into these compositions at least one additional cosmetic agent chosen from those known as conditioning agents, which may be intended mainly to repair or limit the harmful or undesirable effects caused by the various treatments or attacking factors to which the hair fibers may be more or less repeatedly subjected. The at least one additional conditioning agent may, of course, also improve the cosmetic behaviour of natural hair.

With this aim, it may have already been proposed to use cosmetically active organic compounds such as cationic polymers and silicones as conditioning agents in detergent cosmetic compositions such as shampoos, to give the hair at least one satisfactory cosmetic property, for example, chosen from improved sheen, softness, suppleness, lightness, a natural feel and improved disentangling.

However, these compounds in cosmetic washing and hair-conditioning compositions may not give the hair satisfactory and long-lasting styling properties. For example, these compositions may afford styling effects, such as hair hold, body and/or manageability effects, which can remain insufficient and which may have a tendency to fade out after washing the hair with a standard shampoo.

Now, it can be found that consumers may be increasingly in search of washing compositions that can be not only capable of appropriately conditioning the hair, but also capable of affording satisfactory and long-lasting styling effects.

Thus, compositions for washing and conditioning the hair that comprise at least one organosilicon compound, such as 3-aminopropyltriethoxysilane, may have been developed in order to be able to satisfy these requirements. These washing and care compositions may make it possible to condition the hair, for example, by giving it a satisfactory soft feel, while at the same time imparting pronounced and long-lasting styling effects.

Furthermore, these compositions can be beneficial since they may facilitate the shaping of fine hair and can give beneficial styling effects to wavy or curly hair, for example, by improving the fashioning and control of the curls.

However, washing compositions comprising such at least one organosilicon compound may have the drawback of changing substantially over time under normal storage conditions as a function of the temperature, for example, in terms of their viscosity and/or their visual aspect. In other words, these compositions may not be stable, which can be reflected by a cloudy visual aspect and/or by an unsatisfactory texture on storage.

For example, it has been found that organosilicon compounds, such as 3-aminopropyltriethoxysilane, can be chemically incompatible with essentially all the surfactants, for example, anionic surfactants, which may be present in washing compositions, leading to the stability problems encountered.

Moreover, it has been observed that the introduction of certain organosilicon compounds, for example, amino derivatives such as 3-aminopropyltriethoxysilane, into washing compositions that, for example, have a pH ranging from 4 to 7 may also give rise to stability problems due to the alkaline nature of these compounds.

It can be beneficial to develop cosmetic compositions for cleansing and conditioning keratin fibers, comprising at least one organosilicon compound, wherein these compositions do not have at least one of the drawbacks described above, e.g. being stable over time and/or allowing hair to be conditioned satisfactorily while at the same time affording long-lasting, and/or powerful styling effects, for example, in terms of volume, body and/or texturizing of the hair.

Provided herein are detergent and conditioning compositions for keratin fibers, which can have the at least one of beneficial properties discussed above, comprising at least one organosilicon compound as defined hereinbelow, at least one anionic surfactant and at least one nonionic thickener, wherein the weight ratio of the at least one organosilicon compound to the at least one anionic surfactant is equal to or greater than 5×10⁻⁴:1.

For example, it may have been found that the use of at least one nonionic thickener in cosmetic compositions comprising at least one organosilicon compound as defined hereinbelow and at least one anionic surfactant, and the weight ratio of the at least one organosilicon compound to the at least one anionic surfactant being equal to or greater than 5×10⁻⁴:1, can make it possible to render these compositions stable on storage both at room temperature (20-25° C.) and at 45° C., for example, in terms of their visual aspect and/or their viscosity.

As disclosed herein, the term “stable” means that the visual aspect and/or viscosity of these compositions do not change substantially over time under storage test conditions, for example at room temperature (20° C.-25° C.) and/or at 45° C. and/or at 4° C. for two months following their manufacture.

Furthermore, the compositions disclosed herein may lead to satisfactory treatment of the hair, thus giving it a satisfactory soft feel, improved disentangling, softness and/or suppleness.

Moreover, the compositions disclosed herein may afford powerful styling effects, for example, in terms of their provision of volume, body and/or manageability, and/or do so in a lasting manner.

Furthermore, the compositions disclosed herein may facilitate the shaping of the hair, for example, of fine hair, and may give improved styling effects to curly hair, for example, in terms of the fashioning and/or control of the curls, and/or do so in a lasting manner.

Provided herein is a cosmetic composition for washing and conditioning keratin fibers, for example, human keratin fibers such as the hair, comprising, in a cosmetically acceptable medium:

(i) at least one organosilicon compound chosen from silanes comprising one silicon atom and siloxanes comprising two or three silicon atoms, wherein the at least one organosilicon compound also comprises at least one basic chemical functional group and at least one group chosen from hydroxyl and hydrolysable groups per molecule, wherein the at least one organosilicon compound is present in a total amount ranging from 0.01% to 5% by weight, relative to the total weight of the cosmetic composition;

(ii) at least one anionic surfactant, and

(iii) at least one nonionic thickener,

wherein the weight ratio of the at least one organosilicon compound to the at least one anionic surfactant is equal to or greater than 5×10⁻⁴:1.

Also provided is a cosmetic process for treating keratin fibers, for example, a washing and conditioning process, comprising applying the composition disclosed herein to the keratin fibers.

Other subjects and characteristics, aspects and benefits of the present disclosure will emerge even more clearly on reading the description and the examples that follow.

The at least one organosilicon compounds used in the composition disclosed herein can be chosen from organosilanes comprising one silicon atom and organosiloxanes comprising two or three silicon atoms, for example, two silicon atoms. They can also comprise at least one basic chemical functional group, and such as only one basic chemical functional group. The basic chemical functional group may correspond to any functional group that can give the at least one organosilicon compound a basic nature, for example, an amine group such as a primary, secondary or tertiary amine group. The at least one organosilicon compound disclosed herein may optionally comprise at least one additional functional group, for instance, chosen from acid functional groups and halogen.

The at least one organosilicon compound used in the composition disclosed herein also comprises at least one group chosen from hydrolysable and hydroxyl groups per molecule. The hydrolysable groups are, for example, chosen from alkoxy, aryloxy and halogen.

According to at least one embodiment, the at least one organosilicon compound used in the compositions disclosed herein can be chosen from silanes of formula (I):

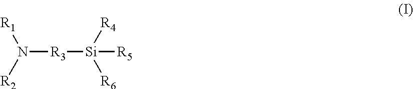

in which:

R₄ represents a halogen, OR′, or R′₁;

R₅ represents a halogen, OR″, R′₂;

R₆ represents a halogen, OR′″, or R′₃;

R₁, R₂, R′, R″, and R′″ represent, independently of each other, hydrogen, or a saturated or unsaturated, linear or branched hydrocarbon-based group optionally bearing at least one additional chemical group;

R³, R′₁, R′₂ and R′₃ represent, independently of each other, a saturated or unsaturated, linear or branched hydrocarbon-based group optionally bearing at least one additional chemical group;

provided that at least two of R₄, R₅ and R₆ respectively represent OR′, OR″ and OR′″, and at least two of R′, R″ and R′″ are not hydrogen.

For example, R₁, R₂, R′, R′₁, R′₂, R′₃, R″ and R′″, independently of each other, are chosen from C₁-C₁₂ alkyl, C₆-C₁₄ aryl, (C₁-C₈)alkyl(C₆-C₁₄)aryl, and (C₆-C₁₄)aryl(C₁-C₈)alkyl radicals.

According to at least one embodiment, the at least one organosilicon compound used in the composition disclosed herein can be chosen from siloxanes of formula (II):

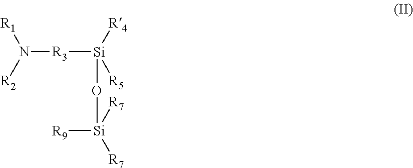

in which:

R₁, R₂, R₃, R₅ and R₆ are as defined previously;

R′₄ represents a halogen, or OR₁₁;

R₇ represents a halogen, OR₁₀, or R″₁;

R₉ represents a halogen, OR₈, R″₂, or R₃NR₁R₂;

R₈, R₁₀ and R₁₁ represent, independently of each other, hydrogen, or a saturated or unsaturated, linear or branched hydrocarbon-based group, optionally bearing at least one additional chemical group;

R″₁ and R″₂ represent, independently of each other, a saturated or unsaturated, linear or branched hydrocarbon-based group, optionally bearing at least one additional chemical group;

provided that at least one of R₆, R₇ and R₉ represents a halogen, OR′″, OR₁₀, or OR₈.

For example, R″₁, R″₂, R₈ or R₁₀ and R₁₁, independently of each other, can be chosen from C₁-C₁₂ alkyl, C₆-C₁₄ aryl, (C₁-C₈)alkyl(C₆-C₁₄)aryl and (C₆-C₁₄)aryl(C₁-C₈)alkyl radicals.

Further for example, the halogen is chlorine.

The at least one organosilicon compound disclosed herein is, for example, chosen from organosilanes of formula (III):

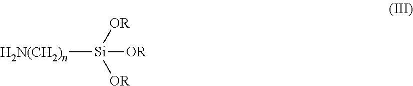

in which the radicals R, which may be identical or different, are chosen from C₁-C₆ such as C₁-C₂ alkyl radicals, and n is an integer ranging from 1 to 6 such as from 2 to 4.

According to at least one embodiment, the at least one silane and/or siloxane are water-soluble such as soluble to a concentration of 2%, further such as soluble to a concentration of 5% and even further such as soluble to a concentration of 10% by weight in water at a temperature of 25° C.±5° C. and at atmospheric pressure. The term “soluble” means the formation of a single macroscopic phase.

According to at least one embodiment, the at least one organosilicon compound present in the composition disclosed herein is 3-aminopropyltriethoxysilane.

The at least one organosilicon compound may be present in the composition disclosed herein in a total amount, for example, ranging from 0.01% to 4.5% by weight, further for example, ranging from 0.1% to 2.5% by weight and even further for example in a total amount ranging from 0.2% to 2% by weight relative to the total weight of the composition.

As indicated previously, the cosmetic composition disclosed herein also comprises at least one anionic surfactant.

The at least one anionic surfactant used in the cosmetic compositions disclosed herein can be, for example, chosen from salts, for example, alkali metal salts such as sodium salts, ammonium salts, amine salts, amino alcohol salts and alkaline-earth metal salts, for example magnesium salts, of the following types: alkyl sulfates, alkyl ether sulfates, alkylamido ether sulfates, alkylaryl polyether sulfates, monoglyceride sulfates, alkylsulfonates, alkylamide sulfonates, alkylarylsulfonates, α-olefin sulfonates, paraffin sulfonates, alkylsulfosuccinates, alkyl ether sulfosuccinates, alkylamide sulfosuccinates, alkylsulfoacetates, acylsarcosinates and acylglutamates, the alkyl and acyl groups of all these compounds comprising from 6 to 24 carbon atoms and the aryl group, for example, representing a phenyl or benzyl group.

It is also possible to use C₆-C₂₄ alkyl monoesters of polyglycoside-dicarboxylic acids such as alkyl glucoside citrates, alkyl polyglycoside tartrates and alkyl polyglycoside sulfosuccinates, alkylsulfosuccinamates, acylisethionates and N-acyltaurates, the alkyl or acyl group of all these compounds comprising from 12 to 20 carbon atoms.

Another group of anionic surfactants that may be used in the compositions disclosed herein is that of acyllactylates in which the acyl group comprises from 8 to 20 carbon atoms.

In addition, exemplary mention may also be made of alkyl-D-galactoside uronic acids and salts thereof and also polyoxyalkylenated (C₆₋₂₄ alkyl) ether carboxylic acids, polyoxyalkylenated (C₆₋₂₄ alkyl)(C₆₋₂₄ aryl) ether carboxylic acids, polyoxyalkylenated (C₆₋₂₄ alkyl)amido ether carboxylic acids and salts thereof, such as those comprising from 2 to 50, further such as from 2 to 10 and even further such as from 2 to 5 ethylene oxide units, and mixtures thereof.

According to at least one embodiment, the at least one anionic surfactant is chosen from alkyl sulfates and alkyl ether sulfates, for example in the form of alkali metal, alkaline-earth metal, ammonium, amine and amino alcohol salts.

According to at least one embodiment, the at least one anionic surfactant used in the cosmetic composition disclosed herein can be chosen from alkyl ether sulfates, such as C₁₂-C₁₄ and for example, comprising from 2 to 3 mol of ethylene oxide, and N-acyltaurates.

The at least one anionic surfactant may be present in a total amount ranging from 1% to 25% by weight, such as in a total amount ranging from 3% to 20% by weight and further such as in a total amount ranging from 5% to 15% by weight relative to the total weight of the cosmetic composition disclosed herein.

As indicated previously, the cosmetic composition disclosed herein also comprises at least one nonionic thickener.

As disclosed herein, the term “thickener” means any compound whose presence can increase the viscosity of the composition into which it is introduced by at least 25 cps and such as by at least 50 cps at 25° C. and at a shear rate of 1 s⁻¹.

The at least one nonionic thickener may be chosen from fatty acid amides, oxyalkylenated fatty acid esters and nonionic thickening polymers.

As disclosed herein, the term “fatty acid amide” means an amide comprising in its structure at least one hydrocarbon-based chain comprising at least 6 carbon atoms.

The fatty acid amides are for example chosen from compounds derived from an amide of an alkanolamine and of a saturated or unsaturated, linear or branched C₈-C₃₀ fatty acid, the alkanolamine and/or the fatty acid being optionally oxyalkylenated and for example being optionally oxyethylenated with 1 to 50 mol of ethylene oxide.

The fatty acid amides are for example chosen from amides of a C₂-C₁₀ alkanolamine and of a C₁₄-C₃₀ fatty acid, for example from amides of a C₂-C₁₀ alkanolamine and of a C₁₄-C₂₂ fatty acid.

For example, the fatty acid amide can be chosen from:
coconut acid monoisopropanolamide, such as the amide sold under the trade name EMPILAN CLS by the company Huntsman,
oleic acid diethanolamide, such as the amide sold under the trade name MEXANYL® GT by the company Chimex,
myristic acid monoethanolamide, such as the amide sold under the trade name COMPERLAN® MM by the company Cognis,
soybean fatty acid diethanolamide, such as the amide sold under the trade name COMPERLAN® VOD by the company Cognis,
stearic acid ethanolamide, such as the amide sold under the trade name MONAMID® S by the company Uniqema,
oleic acid monoisopropanolamide, such as the amide sold under the trade name WITCAMIDE® 61 by the company Witco,
linoleic acid diethanolamide, such as the amide sold under the trade name PURTON® SFD by the company Zschimmer Schwarz,
stearic acid monoethanolamide, such as the amide sold under the trade name MONAMID® 972 by the company ICI/Uniqema,
behenic acid monoethanolamide, such as the amide sold under the trade name INCROMIDE® BEM from Croda,
isostearic acid monoisopropanolamide, such as the amide sold under the trade name WITCAMIDE® SPA by the company Witco,
erucic acid diethanolamide, such as the amide sold under the trade name ERUCIC ACID DIETHANOLAMIDE by the company Stéarineries Dubois,
ricinoleic acid monoethanolamide, such as the amide sold under the trade name RICINOLEIC MONOETHANOLAMIDE by the company Stéarineries Dubois,
rapeseed fatty acid amide comprising 4 mol of ethylene oxide, such as the product sold under the name AMIDET N by the company Kao.

The at least one nonionic thickener may be chosen from oxyalkylenated derivatives of fatty acid esters and of fatty alcohol ethers.

Oxyalkylenated derivatives of fatty acid esters and of fatty alcohol ethers that may, for example, be mentioned include ethoxylated alkyl or acyl derivatives of polyols, which may for example be oxyethylenated derivatives of C₆-C₃₀ fatty acid esters or of C₆-C₃₀ fatty alcohol ethers, and of polyols such as glycerol, sorbitol, glucose, pentaerythritol or polyethylene glycol, further for example, polyethylene glycol, these oxyethylenated derivatives, for example, comprising from 50 to 500 oxyethylene groups such as from 100 to 300 oxyethylene groups.

Examples of compounds of this type that may be mentioned include ethylene glycol stearate, polyethylene glycol distearate comprising 150 oxyethylene groups (150 OE), oxyethylenated (200 OE) glyceryl stearate, such as the product sold under the name SIMULSOL 220 TM® by the company SEPPIC, oxyethylenated (150 OE) pentaerythrityl tetrastearate, such as the product sold under the name CROTHIX® by the company Croda, oxyethylenated (120 OE) methylglucose dioleate, such as the product sold under the name GLUCAMATE DOE-120 VEGETAL® by the company Amerchol, oxyethylenated (160 OE) sorbitan triisostearate, such as the product sold under the name RHEODOL TW IS399C by the company Kao Chemicals, and oxyethylenated (55 ethylene oxide) propylene glycol oleate, such as the product sold under the reference ANTIL 141 Liquid by the company Evonik Goldschmidt, and mixtures thereof.

According to at least one embodiment, the oxyethylenated fatty acid esters are of formula (A):

R₁—CO(X)ₙ—(OCH₂CH₂)ₘ—O—(CO)ₚ—R₂ (A)

in which

X represents a linear or branched C₁-C₄ alkylene radical, and for example the radical having the following formula:

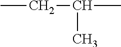

n represents 0 or 1,

p represents 0 or 1,

m represents an integer ranging from 50 to 200, and

R₁ represents a linear or branched C₉-C₂₉ alkyl or alkenyl radical and R₂ represents a hydrogen or a linear or branched C₉-C₂₉ alkyl or alkenyl radical.

For example, among these esters, polyethylene glycol distearate comprising 150 oxyethylene groups (150 OE) may be mentioned.

The nonionic thickening polymers of the disclosure can be different from the amides and esters already described and also from products resulting merely from the condensation of an alkylene oxide with an alcohol, an ester or an amide.

The nonionic thickening polymers may be associative or non-associative polymers.

As disclosed herein, the term “non-associative thickening polymer” means a thickening polymer not simultaneously comprising at least one C₈-C₃ fatty chain and at least one hydrophilic unit.

The nonionic non-associative thickening polymers disclosed herein may be of natural or synthetic origin. They can be chosen, for example, from:

(i) nonionic homopolymers and copolymers comprising ethylenically unsaturated monomers of ester and/or amide type,

(ii) vinylpyrrolidone homopolymers and copolymers, and

(iii) polysaccharides.

Among the nonionic homopolymers and copolymers comprising ethylenically unsaturated monomers of ester and/or amide type that may be mentioned are, for example, polyamides, such as the products sold under the names: CYANAMER P250 by the company Cytec (polyacrylamide); methyl methacrylate/ethylene glycol dimethacrylate copolymers (for example, PMMA MBX-8C by the company US Cosmetics); butyl methacrylate/methyl methacrylate copolymers (for example, ACRYLOID B66 by the company Röhm & Haas); polymethyl methacrylate (for example, BPA 500 by the company Kobo).

The vinylpyrrolidone homopolymers and copolymers can be chosen, for example, from crosslinked vinylpyrrolidone homopolymers such as the polymer ACP-10 sold by ISP.

The nonionic thickening polysaccharides are, for example, chosen from glucans, modified and unmodified starches (such as those derived, for example, from cereals, for instance wheat, corn or rice, from vegetables, for instance yellow pea, and tubers, for instance potato or cassava), amylose, amylopectin, glycogen, dextrans, celluloses and derivatives thereof (methylcelluloses, hydroxyalkylcelluloses, ethylhydroxyethylcelluloses), mannans, xylans, lignins, arabans, galactans, galacturonans, chitin, chitosans, glucuronoxylans, arabinoxylans, xyloglucans, glucomannans, pectic acids and pectins, arabinogalactans, carrageenans, agars, gum arabics, gum tragacanths, ghatti gums, karaya gums, carob gums, galactomannans such as guar gums and nonionic derivatives thereof (hydroxypropyl guar), and mixtures thereof.

For example, the compounds of this type that may be used in the present disclosure can be chosen from those described, for example, in Kirk-Othmer's Encyclopedia of Chemical Technology, Third Edition, 1982, volume 3, pp. 896-900, and volume 15, pp. 439-458, in Polymers in Nature by E. A. MacGregor and C. T. Greenwood, published by John Wiley & Sons, Chapter 6, pp. 240-328, 1980, and in Industrial Gums—Polysaccharides and their Derivatives, edited by Roy L. Whistler, Second Edition, published by Academic Press Inc.

Starches, guar gums and celluloses and derivatives thereof, for example, can be used.

The polysaccharides can be modified or unmodified.

The unmodified guar gums are, for example, the products sold under the name VIDOGUM GH 175 by the company Unipectine and under the names MEYPRO GUAR 50 and JAGUAR C by the company Rhodia Chimie.

The modified nonionic guar gums are, for example, modified with at least one C₁-C₆ hydroxyalkyl group.

Among the hydroxyalkyl groups that may be mentioned, for example, are hydroxymethyl, hydroxyethyl, hydroxypropyl and hydroxybutyl groups.

These guar gums can be prepared, for example, by reacting the corresponding alkene oxides such as, for example, propylene oxides, with the guar gum so as to obtain a guar gum modified with at least one hydroxypropyl group.

The degree of hydroxyalkylation, which corresponds to the number of alkylene oxide molecules consumed by the number of free hydroxyl functions present on the guar gum, for example, ranges from 0.4 to 1.2.

Such nonionic guar gums optionally modified with at least one hydroxyalkyl group are sold, for example, under the trade names JAGUAR HP8, JAGUAR HP60 and JAGUAR HP120, JAGUAR DC 293 and JAGUAR HP 105 by the company Rhodia Chimie or under the name GALACTASOL 4H4FD2 by the company Aqualon.

Among the celluloses, exemplary mention may be made of hydroxyethylcelluloses and hydroxypropylcelluloses. Further exemplary mention may be made of the products sold under the names KLUCEL EF, KLUCEL H, KLUCEL LHF, KLUCEL MF and KLUCEL G by the company Aqualon, and CELLOSIZE POLYMER PCG-10 by the company Amerchol.

The associative nonionic thickening polymers according to the disclosure may be chosen from:
(1) celluloses modified with at least one group comprising at least one fatty chain; of which, examples that may be mentioned include:
   hydroxyethylcelluloses modified with at least one group comprising at least one fatty chain, for example, chosen from alkyl, arylalkyl and alkylaryl groups, and in which the alkyl groups are for example C₈-C₂₂, for instance the product NATROSOL PLUS GRADE 330 CS® (C₁₆ alkyl) sold by the company Aqualon, or the product BERMOCOLL EHM 100® sold by the company Berol Nobel,
   hydroxyethylcelluloses modified with at least one alkylphenyl polyalkylene glycol ether group, such as the product AMERCELL POLYMER HM-1500® (nonylphenyl polyethylene glycol (15) ether) sold by the company Amerchol.
(2) hydroxypropyl guars modified with at least one group comprising at least one fatty chain, such as the product ESAFLOR HM 22® (C₂₂ alkyl chain) sold by the company Lamberti, and the products RE210-18® (C₁₄ alkyl chain) and RE205-1® (C₂₀ alkyl chain) sold by the company Rhône-Poulenc.
(3) copolymers of vinylpyrrolidone and of fatty-chain hydrophobic monomers, of which, examples that may be mentioned include:
   the products ANTARON V216® or GANEX V216® (vinylpyrrolidone/hexadecene copolymer) sold by the company ISP.
   the products ANTARON V220® or GANEX V220® (vinylpyrrolidone/eicosene copolymer) sold by the company ISP.
(4) copolymers of C₁-C₆ alkyl methacrylates or acrylates and of amphiphilic monomers comprising at least one fatty chain, such as, for example, the oxyethylenated methyl acrylate/stearyl acrylate copolymer sold by the company Goldschmidt under the name ANTIL 208®.
(5) copolymers of hydrophilic methacrylates or acrylates and of hydrophobic monomers comprising at least one fatty chain, such as, for example, the polyethylene glycol methacrylate/lauryl methacrylate copolymer.
(6) polyurethane polyethers comprising in their chain both hydrophilic blocks usually of polyoxyethylenated nature and hydrophobic blocks, which may be aliphatic sequences alone and/or cycloaliphatic and/or aromatic sequences.
(7) polymers with an aminoplast ether backbone bearing at least one fatty chain, such as the PURE THIX® compounds sold by the company Sud-Chemie.

For example, the polyurethane polyethers comprise at least two hydrocarbon-based lipophilic chains comprising from 8 to 30 carbon atoms, separated by a hydrophilic block, the hydrocarbon-based chains possibly being pendent chains or chains at the end of the hydrophilic block.

Further for example, it is possible for at least one pendent chain to be included.

In addition, the polyurethane polyethers may comprise a hydrocarbon-based chain at one end or at both ends of a hydrophilic block.

The polyurethane polyethers may be in multiblock, such as in triblock form. Hydrophobic blocks may be at each end of the chain (for example: triblock copolymer with a hydrophilic central block) or distributed both at the ends and within the chain (for example: multiblock copolymer). These same polymers may also be graft polymers or star polymers.

The fatty-chain nonionic polyurethane polyethers may be triblock copolymers in which the hydrophilic block is a polyoxyethylenated chain comprising from 50 to 1000 oxyethylene groups. The nonionic polyurethane polyethers may comprise a urethane linkage between the hydrophilic blocks, whence arises the name.

By extension, also included among the fatty-chain nonionic polyurethane polyethers are those in which the hydrophilic blocks are linked to the lipophilic blocks via other chemical bonds.

As examples of fatty-chain nonionic polyurethane polyethers that may be used in the disclosure, it is also possible to use RHEOLATE 205® comprising a urea function, sold by the company Rheox, or RHEOLATE® 208, 204 or 212, and also ACRYSOL RM 184®.

Exemplary mention may also be made of the product ELFACOS T210® comprising a C₁₂-C₁₄ alkyl chain, and the product ELFACOS T212® comprising a C₁₈ alkyl chain, from Akzo.

The product DW 1206B® from Röhm & Haas comprising a C₂₀ alkyl chain and a urethane linkage, sold at a solids content of 20% in water, may also be used.

It is also possible to use solutions or dispersions of these polymers, for example, in water or in aqueous-alcoholic medium. Examples of such polymers that may be mentioned are RHEOLATE® 255, RHEOLATE® 278 and RHEOLATE® 244 sold by the company Rheox. The products DW 1206F and DW 1206J sold by the company Röhm & Haas may also be used.

The polyurethane polyethers that may be used according to the disclosure are, for example, those described in the article by G. Fonnum, J. Bakke and Fk. Hansen—Colloid Polym. Sci 271, 380.389 (1993).

According to at least one embodiment, a polyurethane polyether that may be obtained by polycondensation of at least three compounds comprising (i) at least one polyethylene glycol comprising from 150 to 180 mol of ethylene oxide, (ii) stearyl alcohol or decyl alcohol, and (iii) at least one diisocyanate, can be used

Such polyurethane polyethers are sold, for example, by the company Röhm & Haas under the names ACULYN 46® and ACULYN 44® [ACULYN 46® is a polycondensate of polyethylene glycol comprising 150 or 180 mol of ethylene oxide, of stearyl alcohol and of methylenebis(4-cyclohexyl isocyanate) (SMDI), at 15% by weight in a matrix of maltodextrin (4%) and water (81%); ACULYN 44® is a polycondensate of polyethylene glycol comprising 150 or 180 mol of ethylene oxide, of decyl alcohol and of methylenebis(4-cyclohexylisocyanate) (SMDI), at 35% by weight in a mixture of propylene glycol (39%) and water (26%)].

According to at least one embodiment, the at least one nonionic thickener can be chosen from the fatty acid amides and oxyethylenated fatty acid esters described above.

The at least one nonionic thickener may be present in a total amount ranging from 0.1% to 20% by weight, such as in a total amount ranging from 0.1% to 10% by weight and further such as in a total amount ranging from 0.2% to 5% by weight relative to the total weight of the composition.

As indicated previously, the weight ratio of the at least one organosilicon compound to the at least one anionic surfactant is equal to or greater than 5×10⁻⁴:1.

For example, the weight ratio of the at least one organosilicon compound to the at least one anionic surfactant ranges from 0.001 to 1, such as from 0.01 to 0.8 and further such as from 0.02 to 0.5.

The compositions disclosed herein may also comprise at least one additional surfactant chosen from amphoteric surfactants and nonionic surfactants.

The amphoteric or zwitterionic surfactants that may be used in the present disclosure may, for example, be secondary or tertiary aliphatic amine derivatives in which the aliphatic group is a linear or branched chain comprising from 8 to 22 carbon atoms and comprising at least one anionic group, for instance a carboxylate, sulfonate, sulfate, phosphate or phosphonate group. Mention may be made, for example, of (C₈-C₂₀)alkylbetaines, sulfobetaines, (C₈-C₂₀)alkylamido(C₆-C₈)alkylbetaines or (C₈-C₂₀)alkylamido(C₆-C₈)alkylsulfobetaines.

Among the amine derivatives, exemplary mention may be made of the products sold under the name MIRANOL®, as described, for example, in U.S. Pat. No. 2,528,378 and U.S. Pat. No. 2,781,354 and classified in the CTFA dictionary, 3rd edition, 1982, under the names Amphocarboxyglycinate and Amphocarboxypropionate, having the respective structures (IV) and (V):

Rₐ—CONHCH₂CH₂—N(R_{b})(R_{c})(CH₂COO⁻) (IV)

in which:

Rₐ represents the alkyl group from an acid Rₐ—COOH present in hydrolysed coconut oil, a heptyl, nonyl or undecyl group,

R_{b} represents a β-hydroxyethyl group, and

R_{c} represents a carboxymethyl group;

and

Rₐ′—CONHCH₂CH₂—N(B)(B′) (V)

in which:

B represents —CH₂CH₂OX′,

B′ represents —(CH₂)_{z}—Y′, with z=1 or 2,

X′ represents —CH₂CH₂—COOH or hydrogen,

Y′ represents —COOH or —CH₂—CHOH—SO₃H,

Rₐ′ represents the alkyl group of an acid Rₐ′—COOH present in coconut oil or in hydrolysed linseed oil, an alkyl group, for example, a C₁₇ alkyl group and its iso form, or an unsaturated C₁₇ group.

These compounds can be classified in the CTFA dictionary, 5th edition, 1993, under the names disodium cocoamphodiacetate, disodium lauroamphodiacetate, disodium caprylamphodiacetate, disodium caprylamphodiacetate, disodium cocoamphodipropionate, disodium lauroamphodipropionate, disodium caprylamphodipropionate, disodium caprylamphodipropionate, lauroamphodipropionic acid, or cocoamphodipropionic acid.

By way of example, mention may be made of the cocoamphodiacetate sold by the company Rhodia under the trade name MIRANOL® C2M Concentrate.

The at least one amphoteric or zwitterionic surfactant may be, for example, chosen from (C₈₋₂₀ alkyl)betaines and (C₈₋₂₀ alkyl)amido(C₆₋₈ alkyl)betaines.

The at least one amphoteric or zwitterionic surfactant can be present in a total mount ranging from 0.1% to 15% by weight and such as from 0.5% to 10% by weight relative to the total weight of the composition disclosed herein.

Examples of additional nonionic surfactants that may be used in the compositions of the present disclosure are described, for example, in the Handbook of Surfactants by M. R. Porter, published by Blackie & Son (Glasgow and London), 1991, pp. 116-178. They can be chosen, for example, from polyethoxylated, polypropoxylated and polyglycerolated fatty acids, (C₁-C₂₀)alkylphenols, α-diols and alcohols, having a fatty chain comprising, for example, 8 to 18 carbon atoms, the number of ethylene oxide or propylene oxide groups possibly ranging for example from 2 to 50 and the number of glycerol groups possibly ranging for example from 2 to 30.

Mention may also be made, for example, of condensates of ethylene oxide and/or of propylene oxide with fatty alcohols; polyethoxylated fatty amides for example having from 2 to 30 ethylene oxide units, polyglycerolated fatty amides comprising on average 1 to 5, and for example from 1.5 to 4, glycerol groups, ethoxylated fatty acid esters of sorbitan comprising from 2 to 30 ethylene oxide units, fatty acid esters of sucrose, fatty acid esters of polyethylene glycol, (C₆₋₂₄ alkyl)polyglycosides, N—(C₆₋₂₄ alkyl)glucamine derivatives, amine oxides such as (C₁₀-C₁₄)alkylamine oxides or N—(C₁₀₋₁₄ acyl)aminopropylmorpholine oxides.

The at least one additional nonionic surfactant may be present in the compositions disclosed herein in a total amount ranging from 0.01% to 20% by weight and such as from 0.1% to 10% by weight relative to the total weight of the composition.

According to at least one embodiment, the total amount of the surfactants in the cosmetic composition disclosed herein may range from 3% to 50% by weight, such as from 5% to 30% by weight and further such as from 8% to 20% by weight relative to the total weight of the cosmetic composition.

The cosmetic composition may also comprise at least cationic polymer.

As disclosed herein, the term “cationic polymer” means any polymer comprising at least one cationic group and/or at least one group that may be ionized into a cationic group.

The at least one cationic polymer that may be present in the composition disclosed herein may be chosen from those already known for improving the cosmetic properties of the hair, e.g. for example, those described in patent application EP-A-337 354 and in French patents FR-2 270 846, 2 383 660, 2 598 611, 2 470 596 and 2 519 863.

The at least one cationic polymer can be chosen, for example, from those comprising at least one primary, secondary, tertiary and/or quaternary amine group, which may either form part of the main polymer chain or be bome by a side substituent directly attached thereto.

The at least one cationic polymer may, for example, have a number-average molecular mass ranging from 500 to 5×10⁶ and such as from 10³ to 3×10⁶.

Among the cationic polymers that may be mentioned, are, for example, polymers of the polyamine, polyamino amide and polyquaternary ammonium type.

These are known products. They are described, for example, in French patents 2 505 348 and 2 542 997. Among the polymers, exemplary mention may be made of:

(1) Homopolymers or copolymers derived from acrylic or methacrylic esters or amides and comprising at least one unit chosen from formula (VI), (VII), (VIII) and (IX) below:

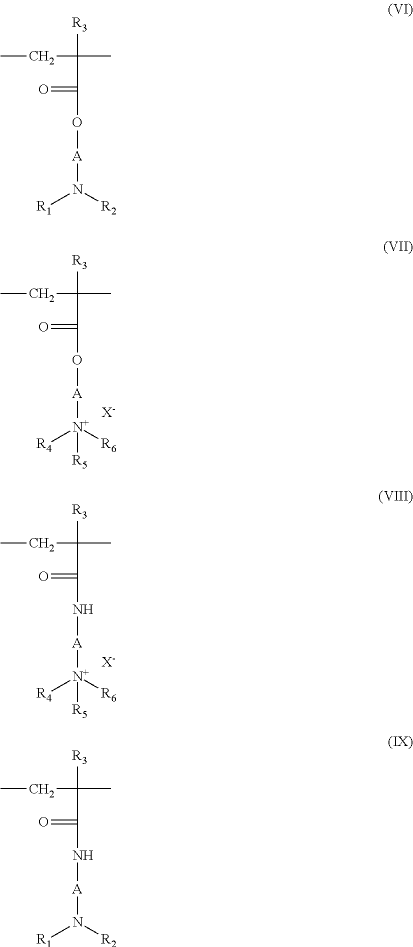

in which:

R₃, which may be identical or different, represents a hydrogen or a CH₃ radical;

A, which may be identical or different, represents a linear or branched alkyl group comprising from 1 to 6 carbon atoms, such as 2 or 3 carbon atoms, or a hydroxyalkyl group comprising from 1 to 4 carbon atoms;

R₄, R₅ and R₆, which may be identical or different, represent an alkyl group comprising from 1 to 18 carbon atoms such as an alkyl group comprising from 1 to 6 carbon atoms, or a benzyl radical;

R₁ and R₂, which may be identical or different, represent hydrogen or an alkyl group comprising from 1 to 6 carbon atoms, such as methyl or ethyl;

X⁻ represents an anion derived from an inorganic or organic acid, such as a methosulfate anion or a halide such as chloride or bromide.

Mention may be made, for example, of ethyltrimethylammonium methacrylate chloride homopolymer.

The polymers of family (1) can also comprise at least one unit derived from comonomers which may be chosen from the family of acrylamides, methacrylamides, diacetone acrylamides, acrylamides and methacrylamides substituted on the nitrogen with lower (C₁-C₄) alkyls, acrylic and methacrylic acids and esters thereof, vinyllactams such as vinylpyrrolidone and vinylcaprolactam, and vinyl esters.

Thus, among these polymers of family (1), exemplary mention may be made of:
copolymers of acrylamide and of dimethylaminoethyl methacrylate quaternized with dimethyl sulfate or with a dimethyl halide, such as the product sold under the name HERCOFLOC by the company Hercules,
the copolymers of acrylamide and of methacryloyloxyethytrimethylammonium chloride described, for example, in patent application EP-A-080 976 and sold under the name BINA QUAT P 100 by the company Ciba Geigy,
the copolymer of acrylamide and of methacryloyloxyethyltnmethylammonium methosulfate sold under the name RETEN by the company Hercules,
quaternized or non-quaternized vinylpyrrolidone/dialkylaminoalkyl acrylate or methacrylate copolymers, such as the products sold under the name GAFQUAT by the company ISP, such as, for example, GAFQUAT 734 or GAFQUAT 755, or alternatively the products known as COPOLYMER 845, 958 and 937. These polymers are, for example, described in detail in French patents 2 077 143 and 2 393 573,
dimethylaminoethyl methacrylate/vinylcaprolactam/vinylpyrrolidone terpolymers, such as the product sold under the name GAFFIX VC 713 by the company ISP,
vinylpyrrolidone/methacrylamidopropyldimethylamine copolymers sold for example under the name STYLEZE CC 10 by ISP,
quaternized vinylpyrrolidone/dimethylaminopropylmethacrylamide copolymers such as the product sold under the name GAFQUAT HS 100 by the company ISP,
crosslinked methacryloyloxy(C₁-C₄)alkyltri(C₁-C₄)alkylammonium salt polymers such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with a compound comprising at least one olefinic group, such as methylenebisacrylamide. A crosslinked acrylamide/methacryloyloxyethyltrimethylammonium chloride copolymer (20/80 by weight) in the form of a dispersion comprising 50% by weight of the copolymer in mineral oil, for example, can be used. This dispersion is sold, for example, under the name SALCARE® SC 92 by the company Ciba. A crosslinked methacryloyloxyethyltrimethylammonium chloride homopolymer comprising about 50% by weight of the homopolymer in mineral oil or in a liquid ester can also be used. These dispersions are sold, for example, under the names SALCARE® SC 95 and SALCARE® SC 96 by the company Ciba.

(2) The cellulose ether derivatives comprising quaternary ammonium groups, described, for example, in French patent 1 492 597, and such as polymers sold under the names UCARE POLYMER “JR” (JR 400, JR 125 and JR 30M) or “LR” (LR 400 or LR 30M) by the company Amerchol. These polymers are, for example, also defined in the CTFA dictionary as quaternary ammoniums of hydroxyethylcellulose that have reacted with an epoxide substituted with a trimethylammonium group.

(3) Cellulose copolymers or cellulose derivatives grafted with a water-soluble monomer of quaternary ammonium, and described, for example, in U.S. Pat. No. 4,131,576, such as hydroxyalkylcelluloses, for instance hydroxymethyl-, hydroxyethyl- or hydroxypropylcelluloses grafted, for example, with a methacryloylethyltrimethylammonium, methacrylamidopropyltrimethylammonium and/or dimethyldiallylammonium salt.

The commercial products corresponding to this definition are for example the products sold under the names CELQUAT L 200 and CELQUAT H 100 by the company National Starch.

(4) The cationic guar gums described for example in U.S. Pat. Nos. 3,589,578 and 4,031,307, such as guar gums comprising cationic trialkylammonium groups. Guar gums modified with a salt (e.g. chloride) of 2,3-epoxypropyltrimethylammonium can be used, for example.

Such products are sold for example under the trade names JAGUAR C13 S, JAGUAR C 15, JAGUAR C 17 or JAGUAR C162 by the company Rhodia.

(5) Polymers formed from piperazinyl units and divalent alkylene or hydroxyalkylene radicals comprising straight or branched chains, optionally interrupted with oxygen, sulfur and/or nitrogen atoms or with aromatic and/or heterocyclic rings, and also the oxidation and/or quaternization products of these polymers. Such polymers are described, for example, in French patents 2 162 025 and 2 280 361.

(6) Water-soluble polyamino amides prepared, for example, by polycondensation of an acidic compound with a polyamine; these polyamino amides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, and/or a bis-alkyl halide, or alternatively with an oligomer resulting from the reaction of a difunctional compound with a bis-halohydrin, a bis-azetidinium, a bis-haloacyldiamine, a bis-alkyl halide, an epihalohydrin, a diepoxide and/or a bis-unsaturated derivative; the crosslinking agent being used in proportions ranging from 0.025 to 0.35 mol per amine group of the polyamino amide; these polyamino amides can be alkylated or, if they comprise at least one tertiary amine functional group, they can be quaternized. Such polymers are described, for example, in French patents 2 252 840 and 2 368 508.

(7) The polyamino amide derivatives resulting from the condensation of polyalkylene polyamines with polycarboxylic acids followed by alkylation with difunctional agents. Mention may be made, for example, of adipic acid/dialkylaminohydroxyalkyl-dialkylenetriamine polymers in which the alkyl radical comprises from 1 to 4 carbon atoms and for example, represents methyl, ethyl or propyl. Such polymers are described, for example, in French patent 1 583 363.

Among these derivatives, mention may be made, for example, of the adipic acid/dimethylaminohydroxypropyl/diethylenetriamine polymers sold under the name CARTARETINE F, F4 or F8 by the company Sandoz.

(8) The polymers obtained by reaction of a polyalkylene polyamine comprising at least two primary amine groups and at least one secondary amine group with a dicarboxylic acid chosen from diglycolic acid and saturated aliphatic dicarboxylic acids comprising from 3 to 8 carbon atoms. The mole ratio of the polyalkylene polyamine to the dicarboxylic acid ranges from 0.8:1 to 1.4:1; the polyamino amide resulting therefrom can be reacted with epichlorohydrin in a mole ratio of epichlorohydrin relative to the secondary amine group of the polyamino amide ranging from 0.5:1 and 1.8:1. Such polymers are described, for example, in U.S. Pat. Nos. 3,227,615 and 2,961,347.

Polymers of this type are sold, for example, under the name HERCOSETT 57 by the company Hercules Inc. or alternatively under the name PD 170 or DELSETTE 101 by the company Hercules in the case of the adipic acid/epoxypropyl/diethylenetriamine copolymer.

(9) Cyclopolymers of alkyldiallylamine or of dialkyldiallylammonium, such as the homopolymers or copolymers comprising, as main constituent of the chain, at least one unit chosen from those of formula (X) and (XI):

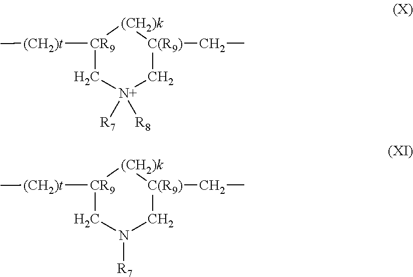

in which k and t are equal to 0 or 1, the sum k+t being equal to 1; R₉ represents hydrogen or a methyl radical; R₇ and R₈, independently of each other, represent an alkyl group comprising from 1 to 6 carbon atoms, a hydroxyalkyl group in which the alkyl group, for example, comprises from 1 to 5 carbon atoms, or a lower (C₁-C₄) amidoalkyl group, or R₇ and R₈ may represent, together with the nitrogen atom to which they are attached, heterocyclic groups, such as piperidyl or morpholinyl; or R₇ and R₈, independently of each other, for example, represent an alkyl group comprising from 1 to 4 carbon atoms; Y is an anion such as bromide, chloride, acetate, borate, citrate, tartrate, bisulfate, bisulfite, sulfate or phosphate. These polymers are described, for example, in French patent 2 080 759 and in its Certificate of Addition 2 190 406.

Among the polymers defined above, mention may be made, for example, of the dimethyldiallylammonium chloride homopolymer sold under the name MERQUAT 100 by the company Nalco (and its homologues of low weight-average molecular mass) and the copolymers of diallyldimethylammonium chloride and of acrylamide, sold under the names MERQUAT 550 and MERQUAT 7SPR.

(10) The quaternary diammonium polymer comprising repeating units of formula (XII):

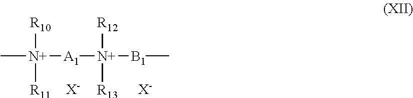

in which:

R₁₀, R₁₁, R₁₂ and R₁₃, which may be identical or different, represent aliphatic, alicyclic or arylaliphatic radicals comprising from 1 to 6 carbon atoms or lower hydroxyalkylaliphatic radicals, or alternatively R₁₀, R₁₁, R₁₂ and R₁₃, together or separately, constitute, with the nitrogen atoms to which they are attached, heterocycles optionally comprising a second heteroatom other than nitrogen, or alternatively R₁₀, R₁₁, R₁₂ and R₁₃ represent, independently of each other, a linear or branched C₁-C₆ alkyl radical optionally substituted with at least one group chosen from nitrile, ester, acyl, amide, —CO—O—R₁₄-D, and —CO—NH—R₁₄-D where R₁₄ is an alkylene and D is a quaternary ammonium group;

A₁ and B₁ represent, independently of each other, polymethylene groups comprising from 2 to 8 carbon atoms, which may be linear or branched, saturated or unsaturated, and which may comprise, linked to or intercalated in the main chain, at least one group chosen from aromatic ring, oxygen, sulfur, sulfoxide, sulfone, disulfide, amino, alkylamino, hydroxyl, quaternary ammonium, ureido, amide, and ester groups, and

X⁻ represents an anion derived from a mineral or organic acid;

A₁, R₁₀ and R₁₂ can form, with the two nitrogen atoms to which they are attached, a piperazine ring; in addition, if A₁ represents a linear or branched, saturated or unsaturated alkylene or hydroxyalkylene radical, B₁ can also represent —(CH₂)ₙ—CO-D-OC—(CH₂)ₙ— group in which D represents:

a) a glycol residue of formula: —O—Z—O—, where Z represents a linear or branched hydrocarbon-based radical or a group corresponding to one of the following formulae:

—(CH₂—CH₂—O)ₓ—CH₂—CH₂—

—[CH₂—CH(CHₐ)—O]_{y}—CH₂—CH(CH₃)—

where x and y represent, independently of each other, an integer ranging from 1 to 4, representing a defined and unique degree of polymerization or any number from 1 to 4 representing an average degree of polymerization;

b) a bis-secondary diamine residue such as a piperazine derivative;

c) a bis-primary diamine residue of formula: —NH—Y—NH—, where Y represents a linear or branched hydrocarbon-based radical, or alternatively the divalent radical

—CH₂—CH₂—S—S—CH₂—CH₂—;

d) a ureylene group of formula: —NH—CO—NH—.

According to at least one embodiment, X is an anion such as chloride or bromide.

These polymers may, for example, have a number-average molecular mass ranging from 1000 to 100,000.

Polymers of this type are described, for example, in French patents 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

It is, for example, possible to use polymers that are formed from repeating units of formula (XIII) below:

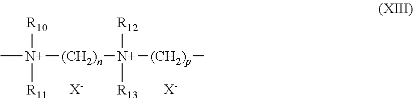

in which R₁₀, R₁₁, R₁₂ and R₁₃, which may be identical or different, represent an alkyl or hydroxyalkyl radical comprising from 1 to 4 carbon atoms, n and p are integers ranging from 2 to 8, and X⁻ is an anion derived from a mineral or organic acid. Mention may be made, for example, of MEXOMER PO sold by the company Chimex.

(11) Polyquaternary ammonium polymers formed from repeating units of formula (XIV):

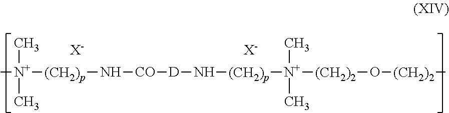

in which p represents an integer ranging from 1 to 6, D may be absent or may represent a group —(CH₂)ᵣCO— in which r represents a number equal to 4 or 7, and X⁻ is an anion.

Such polymers may be prepared according to the processes described, for example, in U.S. Pat. Nos. 4,157,388, 4,702,906 and 4,719,282. They are described, for example, in patent application EP-A-122 324.

Among these products, examples that may be mentioned include MIRAPOL A 15, MIRAPOL AD1, MIRAPOL AZ1 and MIRAPOL 175 sold by the company Miranol.

(12) Quaternary polymers of vinylpyrrolidone and of vinylimidazole, for instance the products sold under the names LUVIQUAT FC 905, FC 550 and FC 370 by the company BASF. These polymers may also comprise other monomers, for instance diallyldialkylammonium halides. Mention may be made, for example, of the product sold under the name LUVIQUAT SENSATION by the company BASF.

(13) Polyamines such as POLYQUART H sold by Henkel, which is given under the reference name Polyethylene glycol (15) Tallow Polyamine in the CTFA dictionary, or oxyethylenated (15 OE) coconut polyamines.

Other cationic polymers that may be used in the context of the disclosure are polyalkyleneimines, such as polyethyleneimines, polymers comprising vinylpyridine and/or vinylpyridinium units, condensates of polyamines and of epichlorohydrin, quaternary polyureylenes and chitin derivatives.

Among all the cationic polymers that may be used in the context of the present disclosure, exemplary mention may be made of polymers of families (1), (2), (4), (9), (10) and (12).

According to at least one embodiment, the at least one cationic polymer is chosen from cationic celluloses, cationic guar gums and quaternary polymers of vinylpyrrolidone and of vinylimidazole optionally combined with other monomers.

According to at least one embodiment, the cationic polymer is chosen from hydroxyalkylcelluloses, such as hydroxymethyl-, hydroxyethyl- or hydroxypropyl-celluloses grafted, for example, with a methacryloylethyltrimethylammonium, methylacrylamidopropyl-trimethylammonium and/or dimethyldiallylammonium salt, cationic guar gums, and the copolymer of vinylpyrrolidone and vinylimidazole and dimethyldiallylammonium chloride.

The at least one cationic polymer may be present in the composition disclosed herein in a total amount ranging from 0.01% to 5% by weight, such as from 0.1% to 1% by weight and further such as from 0.15% to 0.5% by weight relative to the total weight of the composition.

The cosmetic composition disclosed herein may also comprise at least one silicone, such as amino silicones.

As disclosed herein, the term “amino silicone” means any silicone comprising at least one primary, secondary or tertiary amine function or a quaternary ammonium group.

The amino silicones that may be used in the cosmetic composition disclosed herein are chosen from:

(a) compounds of formula (XV) below:

(R¹)ₐ(T)₃₋ₐ—Si[OSi(T)₂]ₙ—[OSi(T)_{b}(R¹)₂₋₅]ₘ—OSi(T)₃₋ₐ—(R¹)ₐ (XV)

in which:

T is a hydrogen or a phenyl, hydroxyl (—OH) or C₁-C₈ alkyl radical, such as methyl, or a C₁-C₈ alkoxy, such as methoxy,

a represents an integer ranging from 0 to 3, and according to at least one embodiment, a is 0,

b represents 0 or 1, and for example, b represents 1,

m and n are numbers such that the sum (n+m) can range, for example, from 1 to 2000 and further for example from 50 to 150, n may represent a number ranging from 0 to 1999 and such as from 49 to 149, and m may represent a number ranging from 1 to 2000 and such as from 1 to 10;

R¹ is a monovalent radical of formula —C_{q}H_{2q}L in which q is a number ranging from 2 to 8 and L is an optionally quaternized amino group chosen from the following groups:

—N(R²)—CH₂—CH₂—N(R²)₂;

—N(R²)₂;

—N⁺(R²)₃Q⁻;

—N⁺(R²)(H)₂Q⁻;

—N⁺(R²)₂HQ⁻; and

—N(R²)—CH₂—CH₂—N⁺(R²)(H)₂Q⁻,

in which R² can represent a hydrogen, a phenyl, a benzyl or a saturated monovalent hydrocarbon-based radical, for example a C₁-C₂₀ alkyl radical, and Q⁻ represents a halide ion such as, for example, fluoride, chloride, bromide or iodide.

For example, the amino silicones of formula (XV) can be chosen from the compounds of formula (XVI):

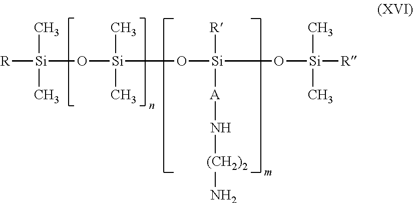

in which R, R′ and R″, which may be identical or different, represent a C₁-C₄ alkyl radical, such as CH₃; a C₁-C₄ alkoxy radical, such as methoxy; or OH; A represents a linear or branched, C₃-C₈ and for example C₃-C₆ alkylene radical; m and n are integers dependent on the molecular weight and whose sum ranges from 1 to 2000.

According to at least one embodiment, R, R′ and R″, which may be identical or different, represent a C₁-C₄ alkyl or hydroxyl radical, A represents a C₃ alkylene radical and m and n are such that the weight-average molecular mass of the compound ranges from 5000 to 500,000. Compounds of this type, for example, are referred to in the CTFA dictionary as Amodimethicones.

According to at least one embodiment, R, R′ and R″, which may be identical or different, represent a C₁-C₄ alkoxy or hydroxyl radical, at least one of R and R″ is an alkoxy radical and A represents a C₃ alkylene radical. The hydroxyl:alkoxy mole ratio, for example, may ranges from 0.2:1 to 0.4:1 and such as equal to 0.3:1. Moreover, m and n are such that the weight-average molecular mass of the compound ranges from 2000 to 1,000,000. For example, n ranges from 0 to 999 and m ranges from 1 to 1000, the sum of n and m being from 1 to 1000.

In this category of compounds, mention may be made, inter alia, of the product BELSIL® ADM 652 sold by Wacker.

According to at least one embodiment, R and R″, which are different, represent a C₁-C₄ alkoxy or hydroxyl radical, at least one of R and R″ is an alkoxy radical, R′ represents a methyl radical and A represents a C₃ alkylene radical. The hydroxyl:alkoxy mole ratio, for example, may range from 1:0.8 to 1:1.1 and is, in at least one embodiment, for example equal to 1:0.95. Moreover, m and n are such that the weight-average molecular mass of the compound ranges from 2000 to 200,000. For example, n ranges from 0 to 999 and m ranges from 1 to 1000, the sum of n and m being from 1 to 1000.

For example, mention may be made of the product FLUID WR® 1300 sold by Wacker.

As disclosed herein, the molecular mass of these silicones can be determined by gel permeation chromatography (ambient temperature, polystyrene standard; μ styragem columns; eluent THF; flow rate 1 mm/m; 200 μl of a solution containing 0.5% by weight of silicone are injected into THF and detection is performed by UV refractometry).

Examples of formula (XV) can be, for example, the polymer known in the CTFA dictionary as Trimethylsilyl Amodimethicone, of formula (XVIII):

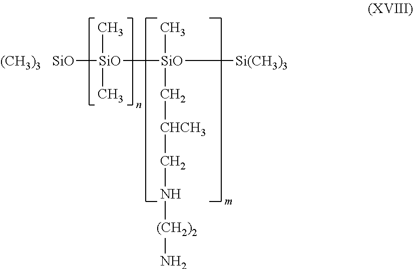

in which n and m have the meanings given above in accordance with formula (XV).

Such compounds are described, for example, in patent application EP 95238; a compound of formula (XVIII) is sold, for example, under the name Q2-8220 by the company OSI.

(b) compounds of formula (XIX):

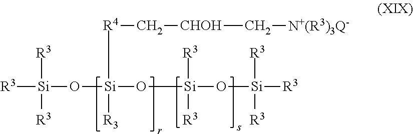

in which:

R³, which may be identical or different, represents a monovalent C₁-C₁₈ hydrocarbon-based radical, and for example a C₁-C₁₈ alkyl or C₂-C₁₈ alkenyl radical, for example methyl;

R⁴ represents a divalent hydrocarbon-based radical, such as a C₁-C₁₈ alkylene radical or a divalent C₁-C₁₈, and for example C₁-C₈, alkyleneoxy radical;

Q⁻ is a halide ion, such as chloride;

r represents an average statistical value ranging from 2 to 20 and such as

from 2 to 8;

s represents an average statistical value ranging from 20 to 200 and such as from 20 to 50.

Such compounds are described, for example, in U.S. Pat. No. 4,185,087.

A compound falling within this class is the product sold by the company Union Carbide under the name UCAR SILICONE ALE 56.

(c) quaternary ammonium silicones of formula (XX):

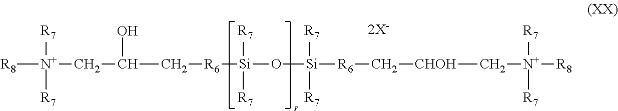

in which:

R₇, which may be identical or different, represents a monovalent hydrocarbon-based radical comprising from 1 to 18 carbon atoms, and for example a C₁-C₁₈ alkyl radical such as methyl, a C₂-C₁₈ alkenyl radical or a ring comprising 5 or 6 carbon atoms;

R₆ represents a divalent hydrocarbon-based radical, for example, a C₁-C₁₈ alkylene radical or a divalent C₁-C₁₈, for example C₁-C₈, alkyleneoxy radical linked to the Si via an Si—C bond;

R₈, which may be identical or different, represent a hydrogen, or a monovalent hydrocarbon-based radical comprising from 1 to 18 carbon atoms, and such as a C₁-C₁₈ alkyl radical, a C₂-C₁₈ alkenyl radical or a —R₆—NHCOR₇ radical;

X⁻ is an anion such as a halide ion, further such as chloride, or an organic acid salt (acetate, etc.);

r represents an average statistical value ranging from 2 to 200 and such as from 5 to 100.

These silicones are described, for example, in patent application EP-A-0 530 974.

(d) the amino silicones of formula (XXI):

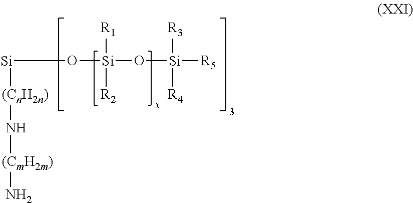

in which:

R₁, R₂, R₃ and R₄, which may be identical or different, represent a C₁-C₄ alkyl radical or a phenyl group,
R₅ represents a C₁-C₄ alkyl radical or a hydroxyl group,
n is an integer ranging from 1 to 5,
m is an integer ranging from 1 to 5, and

in which x is chosen such that the amine number ranges from 0.01 to 1 meq/g.

The term “cosmetically acceptable medium” means a medium that is compatible with keratin fibers, such as the hair.

The cosmetically acceptable medium comprises water or a mixture of water and at least one cosmetically acceptable solvent chosen from C₁-C₄ lower alcohols, such as ethanol, isopropanol, tert-butanol and n-butanol; polyols such as glycerol, propylene glycol and polyethylene glycols.

The pH of the compositions disclosed herein may range from 3 to 11, such as from 5 to 10 and further such as from 7 to 10.

The composition disclosed herein may also comprise at least one standard additive that may be well known in the art, for example, chosen from natural and synthetic thickeners and viscosity regulators other than the nonionic thickeners of the disclosure; C₁₂-C₃₀ fatty alcohols; ceramides; oily fatty esters such as isopropyl myristate, myristyl myristate, cetyl palmitate and stearyl stearate; mineral, plant or synthetic oils such as α-olefins and palm oil; vitamins and provitamins; amphoteric polymers; pH stabilizers, preserving agents; non-amino silicones; and dyes.

The thickener(s) other than the at least one nonionic thickener of (iii) may be chosen from synthetic thickeners such as crosslinked acrylic acid and acrylamidopropanesulfonic acid homopolymers, for example Carbomer, anionic, cationic and amphoteric associative polymers, such as the polymers sold under the names PEMULEN TR1 or TR2 by the company Goodrich, SALCARE SC90 by the company Ciba, ACULYN 22, 28, 33, 44 or 46 by the company Röhm & Haas, and ELFACOS T210 and T212 by the company Akzo.

A person skilled in the art will take care to select the optional additive(s) and the amount thereof such that they do not harm the properties of the compositions of the present disclosure.

The at least one additive may be present in the composition disclosed herein, for example, in a total amount ranging from 0 to 20% by weight relative to the total weight of the composition.

According to at least one embodiment, the cosmetic compositions disclosed herein are transparent or translucent, e.g., these compositions allow a transmittance at 600 nm of greater than 85%, such as greater than 90% and further such as greater than 94%.

The compositions disclosed herein may be used as shampoos for washing and conditioning the hair, and they are for example applied in this case to wet hair in amounts that are effective for washing them, and the lather generated by massaging or frictioning with the hands may then be removed, after an optional leave-on time, by rinsing with water, the operation possibly being repeated at least one time.

Also provided is a cosmetic process for treating keratin fibers, such as the hair, comprising
applying an effective amount of a composition as described above to the fibers, and optionally
rinsing out the composition after an optional leave-on time.

For example, the cosmetic process for treating keratin fibers is a process of washing and conditioning the keratin fibers, such as the hair.

The examples that follow serve to illustrate the present disclosure without limiting the scope thereof.

### EXAMPLES

### Example 1

Composition A according to the disclosure was prepared from the ingredients indicated in the table below, the amounts of which were expressed as weight percentages of product in the given form, relative to the total weight of the composition.

| | A |
|---|---|
| | according |
| | to the |
| Composition | disclosure |
| Lactic acid | 0.27 |
| Mixture of chloro-5-methyl-2-isothiazolin-4-one-3/ | 0.1 |
| methylisothiazolin-4-one-3/magnesium chloride | |
| and nitrate in aqueous solution ⁽¹⁾ | |
| Polyethoxylated (55 OE) propylene glycol | 0.6 |
| oleate and propylene glycol oleate as a | |
| water-glycol solution ⁽²⁾ | |
| Hydroxyethylcellulose quaternized with | 0.6 |
| 2,3-epoxypropyltrimethylammonium chloride | |
| at 95.5% AM ⁽³⁾ | |
| Polydimethylsiloxane containing aminoethyl | 1 |
| aminoisobutyl and trimethylsiloxy group ⁽⁴⁾ | |
| 3-Aminopropyltriethoxysilane ⁽⁵⁾ | 0.75 |
| Cocoylbetaine as an aqueous solution | 17 |
| comprising 30% AM ⁽⁶⁾ | |
| Oxyethylenated (20 OE) and oxypropylenated | 0.5 |
| (5 OP) cetyl alcohol ⁽⁷⁾ | |
| Lauryl ether carboxylic acid (4.5 OE) | 1 |
| at 90% active material ⁽⁸⁾ | |
| Coconut acid monoisopropanolamide ⁽⁹⁾ | 0.85 |
| Sodium lauryl ether sulfate (2.2 OE) | 16 |
| as an aqueous solution (70% AM) ⁽¹⁰⁾ | |
| pH regulator | qs pH = 9 |
| Fragrance | 0.5 |
| Deionized water | qs 100 g |

⁽¹⁾ sold under the trade name KATHON CG by the company Röhm & Haas
⁽²⁾ sold under the trade name ANTIL 141 Liquid by the company Evonik Goldschmidt
⁽³⁾ sold under the trade name POLYQUAT 400 KC by the company KCI
⁽⁴⁾ sold under the trade name DC 8566 Amino Fluid by the company Dow Corning
⁽⁵⁾ sold under the trade name XIAMETER OFS 6O11 Silane by the company Dow Corning
⁽⁶⁾ sold under he trade name MIRATAINE BB/FLA by the company Rhodia
⁽⁷⁾ sold under he trade name PROCETYL AWS-LQ by the company Croda
⁽⁸⁾ sold under the trade name AKYPO RLM 45 CA by the company Kao
⁽⁹⁾ sold under the trade name EMPILAN CIS by the company Huntsman
⁽¹⁰⁾ sold under the name TEXAPON AOS 225UP by the company Cognis

A composition that was clear and stable over time was obtained.

When applied as a shampoo, composition A afforded satisfactory styling effects: for example, this composition gave satisfactory results in terms of the hair body, volume and/or soft feel.

### Example 2

Composition B according to the disclosure was prepared from the ingredients indicated in the table below, the amounts of which were expressed as weight percentages of product in the given form, relative to the total weight of the composition.

| | B |
|---|---|
| | according |
| | to the |
| Composition | disclosure |
| Lactic acid | 0.52 |
| Dimethylol-1,3-dimethyl-5,5-hydantoin | 0.25 |
| as an aqueous solution ⁽¹⁾ | |
| 3-Aminopropyltriethoxysilane ⁽²⁾ | 1.5 |
| Cocoylbetaine as an aqueous | 15 |
| solution comprising 30% AM ⁽³⁾ | |
| Polyethylene glycol distearate (150 OE) ⁽⁴⁾ | 1.1 |
| Sodium cocoyl methyl taurate | 20 |
| as an aqueous dispersion | |
| containing 30% AM ⁽⁵⁾ | |
| pH regulator | qs pH = 7 |
| Fragrance | 0.5 |
| Deionized water | qs 100 g |

⁽¹⁾ sold under the trade name GLYDANT LTD by the company Lonza
⁽²⁾ sold under the trade name XIAMETER OFS 6011 Silane by the company Dow Corning
⁽³⁾ sold under the trade name MIRATAINE BB/FLA by the company Rhodia
⁽⁴⁾ sold under the trade name KESSCO PEG 6000DS by the company Hamalach Chemicals Arese
⁽⁵⁾ sold under the trade name HOSTAPON CT Pate by the company Clariant

A composition that was clear and stable over time was again obtained.

When applied as a hair conditioner, composition B afforded satisfactory styling effects: for example, this composition gave satisfactory results in terms of the hair body and volume, and/or soft feel.

### Example 3

Composition C according to disclosure was prepared from the ingredients indicated in the table below, the amounts of which were expressed as weight percentages of product in the given form, relative to the total weight of the composition.

| | C |
|---|---|
| | according |
| | to the |
| Composition | disclosure |
| Sodium chloride | 1.44 |
| Lactic acid | 0.27 |
| Ethylene glycol distearate | 1.6 |
| Hydroxypropyl guar | 0.2 |
| trimethylammonium chloride ⁽¹⁾ | |
| Carboxyvinyl polymer ⁽²⁾ | 0.15 |
| 3-Aminopropyltriethoxysilane ⁽³⁾ | 0.75 |
| Hexylene glycol | 0.5 |
| Cocoylbetaine as an aqueous | 6 |
| solution comprising 30% AM ⁽⁴⁾ | |
| Coconut acid monoisopropanolamide ⁽⁵⁾ | 0.7 |
| Sodium lauryl ether sulfate (2.2 OE) as | 17.4 |
| an aqueous solution (70% AM) ⁽⁶⁾ | |
| pH regulator | qs pH = 9 |
| Fragrance | 0.5 |
| Deionized water | qs 100 g |

⁽¹⁾ sold under the trade name JAGUAR C13S by the company Rhodia
⁽²⁾ sold under the trade name CARBOPOL 980 by the company Lubrizol
⁽³⁾ sold under the trade name XIAMETER OFS 6011 Silane by the company Dow Corning
⁽⁴⁾ sold under the trade name MIRATAINE BB/FLA by the company Rhodia
⁽⁵⁾ sold under the trade name EMPILAN CLS by the company Huntsman
⁽⁶⁾ sold under the trade name TEXAPON AOS 225UP by the company Cognis

A composition that was transparent and stable over time was again obtained.

When applied as a shampoo, composition C gave satisfactory results in terms of the hair body and volume, and/or soft feel.

1.-20. (canceled)

21. A cosmetic composition for washing and conditioning keratin fibers, comprising, in a cosmetically acceptable medium:
(i) at least one organosilicon compound chosen from compounds of formula (III): 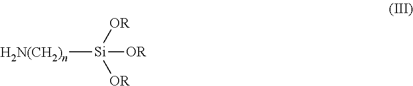
wherein the radicals R, which are identical or different, are chosen from C₁-C₆ alkyl radicals, and n is an integer ranging from 1 to 6, wherein the at least one organosilicon compound is present in a total amount ranging from 0.1% to 2.5% by weight, relative to the total weight of the cosmetic composition;
(ii) at least one anionic surfactant, wherein the anionic surfactant is present in an amount ranging from 1% to 25% by weight, relative to the total weight of the cosmetic composition, and
(iii) at least one nonionic thickener chosen from amides of a C₂-C₁₀ alkanolamine and of a C₁₄-C₂₂ fatty acid, wherein the at least one nonionic thickener is present in an amount ranging from 0.1% to 10% by weight, relative to the total weight of the cosmetic composition,
wherein the weight ratio of the at least one organosilicon compound to the at least one anionic surfactant is equal to or greater than 5×10⁻⁴:1.

22. The cosmetic composition according to claim 21, wherein n is an integer ranging from 2 to 4.

23. The cosmetic composition according to claim 21, wherein the at least one organosilicon compound is 3-aminopropyltriethoxysilane.

24. The cosmetic composition according to claim 21, wherein the at least one organosilicon compound is present in an amount ranging from 0.2% to 2% by weight, relative to the total weight of the composition.

25. The cosmetic composition according to claim 21, wherein the at least one anionic surfactant is chosen from alkyl sulfates, alkyl ether sulfates, or alkyl ether carboxylates.

26. The cosmetic composition according to claim 25, wherein the alkyl sulfates, alkyl ether sulfates, or alkyl ether carboxylates are in the form of alkali metal, alkaline-earth metal, ammonium, amine, or amino alcohol salts.

27. The cosmetic composition according to claim 21, wherein the at least one anionic surfactant is present in an amount ranging from 5% to 15% by weight, relative to the total weight of the composition.

28. The cosmetic composition according to claim 21, wherein the weight ratio of the at least one organosilicon compound to the at least one anionic surfactant ranges from 0.001:1 to 1:1.

29. The cosmetic composition according to claim 21, wherein the weight ratio of the at least one organosilicon compound to the at least one anionic surfactant ranges from 0.002:1 to 0.5:1.

30. The cosmetic composition according to claim 21, wherein the at least one nonionic thickener chosen is present in an amount ranging from 0.2% to 5% by weight, relative to the total weight of the composition.

31. The cosmetic composition according to claim 21, further comprising at least one cationic polymer.

32. The cosmetic composition according to claim 31, wherein the at least one cationic polymer is chosen from polymers of the polyamine, polyamino amide, or polyquaternary ammonium type, cationic celluloses, cationic guar gums, or quaternary polymers of vinylpyrrolidone and of vinylimidazole optionally combined with other monomers.

33. The cosmetic composition according to claim 31, wherein the at least one cationic polymer is present in an amount ranging from 0.01% to 5% by weight, relative to the total weight of the composition.

34. The cosmetic composition according to claim 21, further comprising at least one additional surfactant chosen from amphoteric surfactants, zwitterionic surfactants, or nonionic surfactants.

35. The cosmetic composition according to claim 34, wherein the amphoteric or zwitterionic surfactant is chosen from secondary or tertiary aliphatic amine derivatives wherein the aliphatic group is a linear or branched chain comprising from 8 to 22 carbon atoms and comprising at least one anionic group chosen from carboxylate, sulfonate, sulfate, phosphate, phosphonate, (C₈-C₂₀)alkylbetaines, sulfobetaines, (C₈-C₂₀)alkylamid(C₆-C₈)alkylbetaines, or (C₈-C₂₀)alkylamido(C₆-C₈)alkylsulfobetaines, or the nonionic surfactant is chosen from polyethoxylated, polypropoxylated, or polyglycerolated fatty acids, (C₁-C₂₀)alkylphenols, or α-diols or alcohols.

36. The cosmetic composition according to claim 34, wherein the amphoteric surfactant or zwitterionic surfactant is present in an amount ranging from 0.1% to 15% by weight, relative to the total weight of the composition, or the nonionic surfactant is present in an amount ranging from 0.01% to 20% by weight, relative to the total weight of the composition.

37. The cosmetic composition according to claim 34, wherein the surfactants are present in a total amount ranging from 3% to 50% by weight, relative to the total weight of the composition.

38. The cosmetic composition according to claim 21, further comprising at least one silicone.

39. The cosmetic composition according to claim 38, wherein the at least one silicone is an amino silicone.

40. A cosmetic process for treating keratin fibers, comprising:
applying to the keratin fibers at least one cosmetic composition, and optionally
rinsing out the at least one cosmetic composition after an optional leave-on time,
wherein the at least one cosmetic composition comprises, in a cosmetically acceptable medium:
(i) at least one organosilicon compound chosen from compounds of formula (III): 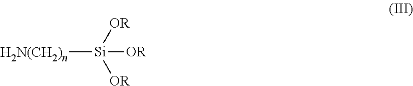
wherein the radicals R, which are identical or different, are chosen from C₁-C₆ alkyl radicals, and n is an integer ranging from 1 to 6, wherein the at least one organosilicon compound is present in a total amount ranging from 0.1% to 2.5% by weight, relative to the total weight of the cosmetic composition;
(ii) at least one anionic surfactant, wherein the anionic surfactant is present in an amount ranging from 1% to 25% by weight, relative to the total weight of the cosmetic composition, and
(iii) at least one nonionic thickener chosen from amides of a C₂-C₁₀ alkanolamine and of a C₁₄-C₂₂ fatty acid, wherein the at least one nonionic thickener is present in an amount ranging from 0.1% to 10% by weight, relative to the total weight of the cosmetic composition,
wherein the weight ratio of the at least one organosilicon compound to the at least one anionic surfactant is equal to or greater than 5×10⁻⁴:1.
